(19) United States
(12) Patent Application Publication
(10) Pub. No.: US 2023/0389985 A1
(43) Pub. Date: Dec. 7, 2023

(54) CATHETER TIP INSULATOR

(71) Applicant: Boston Scientific Scimed, Inc., Maple Grove, MN (US)

(72) Inventors: Trey Henry Achterhoff, Arden Hills, MN (US); Andrew L. De Kock, Ham Lake, MN (US); Nathan Gaworski, St. Paul, MN (US); Andrew Dauwalter, Waconia, MN (US)

(21) Appl. No.: 18/328,487

(22) Filed: Jun. 2, 2023

Related U.S. Application Data
(60) Provisional application No. 63/348,075, filed on Jun. 2, 2022.

(51) Int. Cl. A61B 18/14

(52) U.S. Cl. CPC A61B 18/1492; A61B 18/14; A61B 2018/00577

(57) ABSTRACT

Various aspects of the present disclosure are directed towards apparatuses, systems and methods that may include a cardiac ablation catheter. The cardiac ablation catheter may include a handle, an elongated shaft, and a distal assembly including a tip electrode, a ring electrode, and an insulator preform.

### CROSS REFERENCE TO RELATED APPLICATIONS

The present application claims priority to U.S. Provisional Patent Application No. 63/348,075, filed Jun. 2, 2022, the entire disclosure of which is hereby incorporated in its entirety.

### TECHNICAL FIELD

The present disclosure relates to medical apparatus, systems, and methods for cardiac ablation. More specifically, the present disclosure relates to a point pulsed field ablation catheter.

### BACKGROUND

Ablation procedures are used to treat many different conditions in patients. Ablation may be used to treat cardiac arrhythmias, benign tumors, cancerous tumors, and to control bleeding during surgery. Usually, ablation is accomplished through thermal ablation techniques including radio-frequency (RF) ablation and cryoablation. In RF ablation, a probe is inserted into the patient and radio frequency waves are transmitted through the probe to the surrounding tissue. The radio frequency waves generate heat, which destroys surrounding tissue and cauterizes blood vessels. In cryoablation, a hollow needle or cryoprobe is inserted into the patient and cold, thermally conductive fluid is circulated through the probe to freeze and kill the surrounding tissue.

Another ablation technique uses electroporation. In electroporation, or electro-permeabilization, an electric field is applied to cells to increase the permeability of the cell membrane. The electroporation may be reversible or irreversible, depending on the strength of the electric field. If the electroporation is reversible, the increased permeability of the cell membrane may be used to introduce chemicals, drugs, and/or deoxyribonucleic acid (DNA) into the cell, prior to the cell healing and recovering. If the electroporation is irreversible, the affected cells are killed through apoptosis.

Irreversible electroporation (IRE) may be used as a nonthermal ablation technique. In IRE, trains of short, high voltage pulses are used to generate electric fields that are strong enough to kill cells through apoptosis. In ablation of cardiac tissue, IRE may be a safe and effective alternative to the indiscriminate killing of thermal ablation techniques, such as RF ablation and cryoablation. IRE may be used to kill target tissue, such as myocardium tissue, by using an electric field strength and duration that kills the target tissue but does not permanently damage other cells or tissue, such as non-targeted myocardium tissue, red blood cells, vascular smooth muscle tissue, endothelium tissue, and nerve cells.

There is a continuing need for improved catheter devices for performing IRE procedures.

### SUMMARY

In Example 1, a cardiac ablation catheter comprising an elongated shaft and a distal assembly. The shaft has a proximal end and a distal end. The distal assembly has a proximal end and a distal end, the proximal end secured to the distal end of the shaft. The distal assembly comprises a tip electrode, a first ring electrode and an insulator preform. The tip electrode is located at the distal end of the distal assembly. The first ring electrode is located proximal of and spaced apart from the tip electrode, the first ring electrode having a distal leading end and a proximal trailing end. The an insulator preform comprises a proximal portion having a forward portion defining a first diameter, and a distal portion extending distally from the proximal portion, the distal portion having a distal face and a second diameter greater than the first diameter so as to define a radial shoulder, and a distal portion length. The tip electrode extends distally from the distal face of the insulator preform, and the first ring electrode is disposed over the proximal portion such that the distal leading end of the ring electrode abuts the radial shoulder of the insulator preform, and wherein the distal portion length defines a longitudinal spacing between the tip electrode and the first ring electrode distal leading end.

In Example 2, the cardiac ablation catheter of Example 1, wherein the tip electrode includes a tip electrode shoulder that abuts the distal face of the insulator preform.

In Example 3, the cardiac ablation catheter of either of Examples 1 or 2, wherein the distal portion of the insulator preform includes a distal opening in the distal face.

In Example 4, the cardiac ablation catheter of Example 3, wherein the tip electrode includes an active portion having an active portion diameter, and a tip electrode shank having a tip electrode shank diameter that is smaller than the active portion diameter, and wherein the tip electrode shank is received within the distal opening in the distal face of the insulator preform.

In Example 5, the cardiac ablation catheter of any of Examples 1-4, wherein the distal assembly further comprises a second ring electrode located proximally of and longitudinally spaced from the first ring electrode.

In Example 6, the cardiac ablation catheter of any of Examples 1-5, wherein the distal assembly further comprises an insulating material disposed at least proximally of the first ring electrode.

In Example 7, the cardiac ablation catheter of Example 6, wherein the insulating material is disposed between the first ring electrode and the second ring electrode.

In Example 8, the cardiac ablation catheter of Example 7, wherein the insulating material is formed by an overmolding process.

In Example 9, the cardiac ablation catheter of Example 8, wherein the insulator preform includes first and second longitudinal channels extending through the proximal portion to the insulator preform distal portion.

In Example 10, the cardiac ablation catheter of Example 9, wherein the insulating material extends through the first and second longitudinal channels and about the tip electrode shank so as to secure the tip electrode to the insulator preform.

In Example 11, the cardiac ablation catheter of Example 9, wherein the tip electrode shank includes a plurality of radial projections that abut an inner surface of the insulator preform distal portion, and wherein the insulating material encapsulates the radial projections to secure the tip electrode to the insulator preform.

In Example 12, the cardiac ablation catheter of any of Examples 8-11, wherein the tip electrode shank includes a plurality of radial apertures extending inward.

In Example 13, the cardiac ablation catheter of Example 12, wherein the insulating material extends through the radial apertures to secure the insulator preform to the distal assembly.

In Example 14, the cardiac ablation catheter of any of Examples 1-13, wherein the insulator preform proximal portion includes a proximal opening and a navigator sensor lumen extending from the proximal opening and terminating in blind hole.

In Example 15, the cardiac ablation catheter of any of Examples 1-14, wherein the insulator preform proximal portion has a planar portion defining a space to accommodate attachment of an electrical conductor to the first ring electrode.

In Example 16, a cardiac ablation catheter comprising a handle, an elongated shaft and a distal assembly. The shaft has a proximal end and a distal end, the proximal end extending distally from the handle. The distal assembly has a proximal end and a distal end, the proximal end secured to the distal end of the shaft. The distal assembly comprises a tip electrode, a first ring electrode and an insulator preform. The tip electrode is located at the distal end of the distal assembly. The first ring electrode is located proximal of and spaced apart from the tip electrode, the first ring electrode having a distal leading end and a proximal trailing end. The an insulator preform comprises a proximal portion having a forward portion defining a first diameter, and a distal portion extending distally from the proximal portion, the distal portion having a distal face and a second diameter greater than the first diameter so as to define a radial shoulder, and a distal portion length. The tip electrode extends distally from the distal face of the insulator preform, and the first ring electrode is disposed over the proximal portion such that the distal leading end of the ring electrode abuts the radial shoulder of the insulator preform, and wherein the distal portion length defines a longitudinal spacing between the tip electrode and the first ring electrode distal leading end.

In Example 17, the cardiac ablation catheter of Example 16, wherein the tip electrode includes a tip electrode shoulder that abuts the distal face of the insulator preform.

In Example 18, the cardiac ablation catheter of Example 17, wherein the distal portion of the insulator preform includes a distal opening in the distal face.

In Example 19, the The cardiac ablation catheter of Example 18, wherein the tip electrode includes an active portion having an active portion diameter, and a tip electrode shank having a tip electrode shank diameter that is smaller than the active portion diameter, and wherein the tip electrode shank is received within the distal opening in the distal face of the insulator preform.

In Example 20, the cardiac ablation catheter of Example 19, wherein the distal assembly further comprises a second ring electrode located proximally of and longitudinally spaced from the first ring electrode.

In Example 21, the cardiac ablation catheter of Example 20, wherein the distal assembly further comprises an insulating material disposed at least proximally of the first ring electrode.

In Example 22, the cardiac ablation catheter of Example 21, wherein the insulating material is disposed between the first ring electrode and the second ring electrode.

In Example 23, the cardiac ablation catheter of Example 22, wherein the insulator preform includes first and second longitudinal channels extending through the proximal portion to the insulator preform distal portion.

In Example 24, the cardiac ablation catheter of Example 23, wherein the insulating material extends through the first and second longitudinal channels and about the tip electrode shank so as to secure the tip electrode to the insulator preform.

In Example 25, the cardiac ablation catheter of Example 23, wherein the tip electrode shank includes a plurality of radial projections that abut an inner surface of the insulator preform distal portion, and wherein the insulating material encapsulates the radial projections to secure the tip electrode to the insulator preform.

In Example 26, the cardiac ablation catheter of Example 22, wherein the tip electrode shank includes a plurality of radial apertures extending inward, and wherein the insulating material extends through the radial apertures to secure the insulator preform to the distal assembly.

In Example 27, the cardiac ablation catheter of Example 26, wherein the insulator preform proximal portion includes a proximal opening and a navigator sensor lumen extending from the proximal opening and terminating in blind hole.

In Example 28, the cardiac ablation catheter of Example 27, wherein the insulator preform proximal portion has a planar portion defining a space to accommodate attachment of an electrical conductor to the first ring electrode.

In Example 29, an ablation electrode assembly for a pulsed field ablation catheter, the ablation electrode assembly comprising an insulator preform, a tip electrode and a ring electrode. The insulator preform comprises a proximal portion having a forward portion defining a first diameter, and a distal portion extending distally from the proximal portion, the distal portion having a distal face and a second diameter greater than the first diameter so as to define a radial shoulder, and a distal portion length. The tip electrode extends distally from the distal face of the insulator preform. The ring electrode is disposed over the proximal portion of the insulator preform such that a distal leading end of the ring electrode abuts the radial shoulder of the insulator preform, and wherein the distal portion length of the insulator preform defines a longitudinal spacing between the tip electrode and the first ring electrode distal leading end.

In Example 30, the ablation electrode assembly of Example 29, wherein the tip electrode includes a tip electrode shoulder that abuts the distal face of the insulator preform.

In Example 31, the ablation electrode assembly of 30, wherein the distal portion of the insulator preform includes a distal opening in the distal face.

In Example 32, the ablation electrode assembly of Example 31, wherein the tip electrode includes an active portion having an active portion diameter, and a tip electrode shank having a tip electrode shank diameter that is smaller than the active portion diameter, and wherein the tip electrode shank is received within the distal opening in the distal face of the insulator preform.

In Example 33, the cardiac ablation catheter of Example 29, wherein the insulator preform includes first and second longitudinal channels extending through the proximal portion to the insulator preform distal portion, and wherein an insulating material extends through the first and second longitudinal channels and about the tip electrode shank so as to secure the tip electrode to the insulator preform.

In Example 34, a method of making an ablation electrode assembly of a cardiac ablation catheter, the method comprising providing an insulator preform comprising a proximal portion having a forward portion defining a first diameter, and a distal portion extending distally from the proximal portion, the distal portion having a distal face having a distal opening, a second diameter greater than the first diameter so as to define a radial shoulder, and a distal portion length, securing a tip electrode to the distal portion of the insulator preform so that the tip electrode extends distally from the distal face of the insulator preform, and securing a ring electrode over the proximal portion of the insulator preform such that a distal leading end of the ring electrode abuts the radial shoulder of the insulator preform, wherein the distal portion length of the insulator preform defines a longitudinal spacing between the tip electrode and the first ring electrode distal leading end.

In Example 35, the method of Example 34, wherein the tip electrode includes an active portion having an active portion diameter, and a tip electrode shank having a tip electrode shank diameter that is smaller than the active portion diameter, and wherein securing the tip electrode to the insulator preform includes inserting the tip electrode shank within the distal opening in the distal face of the insulator preform.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a diagram illustrating an exemplary clinical setting for treating a patient, using a cardiac ablation catheter in accordance with embodiments of the subject matter of the disclosure,

FIG. 2 is an isometric illustration of a distal portion of the cardiac ablation catheter depicted in FIG. 1, including a distal assembly according to embodiments of the disclosure,

FIGS. 3A and 3B are isometric and cross-sectional isometric illustrations of an insulator preform used in the distal assembly of the cardiac ablation catheter of FIG. 1, according to embodiments of the present disclosure,

FIG. 3C is a plan view illustration of the insulator preform used in the distal assembly of the cardiac ablation catheter of FIG. 1, according to embodiments of the present disclosure,

FIG. 4 is a cross-sectional elevation view of a portion of the distal assembly of the cardiac ablation catheter depicted in FIG. 1, according to embodiments of the present disclosure,

FIG. 5 is an elevation view of an insulator preform for use in the distal assembly of the cardiac ablation catheter of FIG. 1, according to alternative embodiments of the present disclosure.

FIG. 6 is an elevation view of an insulator preform for use in the distal assembly of the cardiac ablation catheter of FIG. 1, according to alternative embodiments of the present disclosure.

FIG. 7 is an isometric illustration of a tip electrode used in the distal assembly of the cardiac ablation catheter of FIG. 1, according to embodiments of the present disclosure,

FIGS. 8A and 8B are illustrations of an alternative design of a tip electrode used in the distal assembly of the cardiac ablation catheter of FIG. 1, according to embodiments of the present disclosure.

FIG. 9 is a cross-sectional view of another alternative design of a tip electrode used in the distal assembly of the cardiac ablation catheter of FIG. 1, according to embodiments of the present disclosure.

FIG. 10 is an elevation view of a handle of the cardiac ablation catheter of FIG. 1, according to embodiments of the present disclosure.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

The following detailed description is exemplary in nature and is not intended to limit the scope, applicability, or configuration of the invention in any way. Rather, the following description provides some practical illustrations for implementing exemplary embodiments of the present invention. Examples of constructions, materials, and/or dimensions are provided for selected elements. Those skilled in the art will recognize that many of the noted examples have a variety of suitable alternatives.

As the terms are used herein with respect to measurements (e.g., dimensions, characteristics, attributes, components, etc.), and ranges thereof, of tangible things (e.g., products, inventory, etc.) and/or intangible things (e.g., data, electronic representations of currency, accounts, information, portions of things (e.g., percentages, fractions), calculations, data models, dynamic system models, algorithms, parameters, etc.), “about” and “approximately” may be used, interchangeably, to refer to a measurement that includes the stated measurement and that also includes any measurements that are reasonably close to the stated measurement, but that may differ by a reasonably small amount such as will be understood, and readily ascertained, by individuals having ordinary skill in the relevant arts to be attributable to measurement error; differences in measurement and/or manufacturing equipment calibration; human error in reading and/or setting measurements; adjustments made to optimize performance and/or structural parameters in view of other measurements (e.g., measurements associated with other things); particular implementation scenarios; imprecise adjustment and/or manipulation of things, settings, and/or measurements by a person, a computing device, and/or a machine; system tolerances; control loops; machine-learning; foreseeable variations (e.g., statistically insignificant variations, chaotic variations, system and/or model instabilities, etc.); preferences; and/or the like.

Although illustrative methods may be represented by one or more drawings (e.g., flow diagrams, communication flows, etc.), the drawings should not be interpreted as implying any requirement of, or particular order among or between, various steps disclosed herein. However, certain some embodiments may require certain steps and/or certain orders between certain steps, as may be explicitly described herein and/or as may be understood from the nature of the steps themselves (e.g., the performance of some steps may depend on the outcome of a previous step). Additionally, a “set,” “subset,” or “group” of items (e.g., inputs, algorithms, data values, etc.) may include one or more items, and, similarly, a subset or subgroup of items may include one or more items. A “plurality” means more than one.

As used herein, the term “based on” is not meant to be restrictive, but rather indicates that a determination, identification, prediction, calculation, and/or the like, is performed by using, at least, the term following “based on” as an input. For example, predicting an outcome based on a particular piece of information may additionally, or alternatively, base the same determination on another piece of information.

Irreversible electroporation (IRE) uses high voltage, short (e.g., 100 microseconds) pulses to kill cells through apoptosis. IRE can be targeted to kill myocardium, sparing other adjacent tissues including the esophageal vascular smooth muscle and endothelium. Failures of dielectric isolation between ablation poles could lead to therapy energy shunting through the catheter rather than being delivered to target tissue, as well as unintentional arcs or localized high current that may cause damage to the catheter and/or possibly surrounding tissues. Therefore, with the introduction of high voltage therapy used in IRE, the need for robust dielectric isolation between circuits is needed throughout the entire catheter, especially in the tip region of the catheter where therapy is delivered.

Current catheter processes rely on material flow to seal joints and prevent fluid pathways between exposed conductors (i.e., reflowed and adhesive joints). In these processes, voids or bubbles may form and may be difficult to identify.

At least some embodiments of the present disclosure are directed to provide a guaranteed insulation layer between all conductive surfaces and wires at high potential to each other within the tip region of the catheter. In some embodiments, an electroporation ablation system includes a point electroporation ablation catheter with an insulator preform. As used herein, a point catheter refers to a catheter with a linear body carrying ablation electrodes. In embodiments, a point catheter has ablation electrodes toward its distal end.

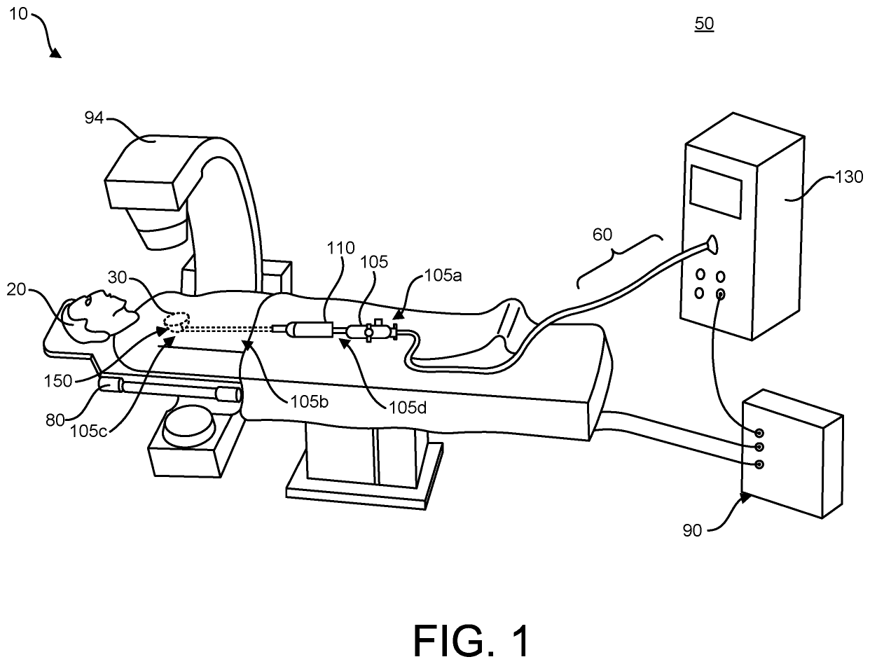

FIG. 1 is a diagram illustrating an exemplary clinical setting 10 for treating a patient 20, and for treating a heart 30 of the patient 20, using an electrophysiology system 50, in accordance with embodiments of the subject matter of the disclosure. The electrophysiology system 50 includes an electroporation device 60 and an optional localization field generator 80. Also, the clinical setting 10 includes additional equipment such as imaging equipment 94 (represented by the C-arm) and various controller elements configured to allow an operator to control various aspects of the electrophysiology system 50. As will be appreciated by the skilled artisan, the clinical setting 10 may have other components and arrangements of components that are not shown in FIG. 1.

The electroporation device 60 includes a cardiac ablation catheter 105, an introducer sheath 110, a controller 90, and an electroporation generator 130. In embodiments, the electroporation device 60 is configured to deliver electric field energy to target tissue in the patient's heart 30 to create tissue apoptosis, rendering the tissue incapable of conducting electrical signals. The controller 90 is configured to control functional aspects of the electroporation device 60. In embodiments, the controller 90 is configured to control the electroporation generator 130 to generate electrical pulses, for example, the magnitude of the electrical pulses, the timing and duration of electrical pulses. In embodiments, the electroporation generator 130 is operable as a pulse generator for generating and supplying pulse sequences to the cardiac ablation catheter 105.

In embodiments, the introducer sheath 110 is operable to provide a delivery conduit through which the cardiac ablation catheter 105 may be deployed to the specific target sites within the patient's heart 30. It will be appreciated, however, that the introducer sheath 110 is illustrated and described herein to provide context to the overall electrophysiology system 50.

In the illustrated embodiment, the cardiac ablation catheter 105 includes a handle 105a, an elongated shaft 105b, and a distal assembly 150. As shown, the shaft has a distal end 105c and a proximal end 105d, and the proximal end 105d of the shaft 105b extends distally from the handle 105a. The handle 105a is configured to be operated by a user to position the distal assembly 150 at the desired anatomical location. The shaft 105b generally defines a longitudinal axis of the cardiac ablation catheter 105. The shaft 105b may include a molded articulation joint for spine reinforcement and steering capability. More details may be found at U.S. Pat. App. 63/129,960, which is hereby incorporated by reference in its entirety.

As shown, the distal assembly 150 is located at or proximate the distal end 105c of the shaft 105b. In embodiments, the distal assembly 150 is electrically coupled to the electroporation generator 130, to receive electrical pulse sequences or pulse trains, thereby selectively generating electrical fields for ablating the target tissue by irreversible electroporation.

In certain embodiments, the cardiac ablation catheter 105 is a point catheter that includes a linear body toward the distal end. In embodiments, the distal assembly 150 includes one or more electrodes disposed on the shaft 105b. In some implementations, the distal assembly 150 includes one or more electrode pairs. In some embodiments, the distal assembly 150 includes one or more ablation electrodes and one or more sensing electrodes. In certain implementations, the distal assembly 150 includes a pair of ablation electrodes configured to generate electrical fields sufficient for irreversible electroporation ablation. In some examples, the ablation electrode pair including a tip electrode covering the distal end of the catheter 105 and a ring electrode disposed proximate to the tip electrode. As used herein, a ring electrode refers to an electrode having a ring shape. In some designs, the pair of ablation electrodes include two ring electrodes disposed proximate to the distal end of the catheter 105.

In embodiments, the electrode positions and sizes are specifically designed to allow flexibility. For example, the electrodes are designed to be relatively short in length. As another example, two electrodes have a relatively larger spacing to allow flexibility and/or deflection. In some examples, the one or more electrodes include one or more pairs of ablation electrodes and one or more pairs of sensing electrodes. The sensing electrodes may be used to sense electrical signals related to a patient's heart, which allows an operator or a system to determine whether ablation has occurred or not. In some designs, the electrical signals can be used to determine a location or proximate location of the cardiac ablation catheter 105. In some embodiments, other sensors, such as force sensors, navigation sensors (e.g., five or six degree-of-freedom (“DoF”) sensors), may be incorporated in the distal assembly 150.

In some embodiments, the one or more sensing electrodes on the cardiac ablation catheter 105 can measure electrical signals and generate output signals that can be processed by a controller (e.g., the controller 90) to generate an electro-anatomical map. In some instances, electro-anatomical maps are generated before ablation for determining the electrical activity of the cardiac tissue within a chamber of interest. In some instances, electro-anatomical maps are generated after ablation in verifying the desired change in electrical activity of the ablated tissue and the chamber as a whole. The sensing electrodes may be used to determine the position of the catheter 105 in three-dimensional space within the body. For example, when the operator moves the catheter 105 within a cardiac chamber of a patient, the boundaries of catheter movement can be determined by the controller 90, which may include or couple to a mapping and navigation system, to form the anatomy of the chamber. The chamber anatomy may be used to facilitate navigation of the catheter 105 without the use of ionizing radiation such as with fluoroscopy, and for tagging locations of ablations as they are completed in order to guide spacing of ablations and aid the operator in fully ablating the anatomy of interest.

According to embodiments, various components (e.g., the controller 90) of the electrophysiological system 50 may be implemented on one or more computing devices. A computing device may include any type of computing device suitable for implementing embodiments of the disclosure. Examples of computing devices include specialized computing devices or general-purpose computing devices such as workstations, servers, laptops, portable devices, desktop, tablet computers, hand-held devices, general-purpose graphics processing units (GPGPUs), and the like, all of which are contemplated within the scope of FIG. 1 with reference to various components of the system 50.

In some embodiments, a computing device includes a bus that, directly and/or indirectly, couples the following devices: a processor, a memory, an input/output (I/O) port, an I/O component, and a power supply. Any number of additional components, different components, and/or combinations of components may also be included in the computing device. The bus represents what may be one or more busses (such as, for example, an address bus, data bus, or combination thereof). Similarly, in some embodiments, the computing device may include a number of processors, a number of memory components, a number of I/O ports, a number of I/O components, and/or a number of power supplies. Additionally, any number of these components, or combinations thereof, may be distributed and/or duplicated across a number of computing devices.

In some embodiments, the system 50 includes one or more memories (not illustrated). The one or more memories includes computer-readable media in the form of volatile and/or nonvolatile memory, transitory and/or non-transitory storage media and may be removable, nonremovable, or a combination thereof. Media examples include Random Access Memory (RAM); Read Only Memory (ROM); Electronically Erasable Programmable Read Only Memory (EEPROM); flash memory; optical or holographic media; magnetic cassettes, magnetic tape, magnetic disk storage or other magnetic storage devices; data transmissions; and/or any other medium that can be used to store information and can be accessed by a computing device such as, for example, quantum state memory, and/or the like. In some embodiments, the one or more memories store computer-executable instructions for causing a processor (e.g., the controller 90) to implement aspects of embodiments of system components discussed herein and/or to perform aspects of embodiments of methods and procedures discussed herein.

Computer-executable instructions may include, for example, computer code, machine-useable instructions, and the like such as, for example, program components capable of being executed by one or more processors associated with a computing device. Program components may be programmed using any number of different programming environments, including various languages, development kits, frameworks, and/or the like. Some or all of the functionality contemplated herein may also, or alternatively, be implemented in hardware and/or firmware.

In some embodiments, the memory may include a data repository implemented using any one of the configurations described below. A data repository may include random access memories, flat files, XML files, and/or one or more database management systems (DBMS) executing on one or more database servers or a data center. A database management system may be a relational (RDBMS), hierarchical (HDBMS), multidimensional (MDBMS), object oriented (ODBMS or OODBMS) or object relational (ORDBMS) database management system, and the like. The data repository may be, for example, a single relational database. In some cases, the data repository may include a plurality of databases that can exchange and aggregate data by data integration process or software application. In an exemplary embodiment, at least part of the data repository may be hosted in a cloud data center. In some cases, a data repository may be hosted on a single computer, a server, a storage device, a cloud server, or the like. In some other cases, a data repository may be hosted on a series of networked computers, servers, or devices. In some cases, a data repository may be hosted on tiers of data storage devices including local, regional, and central.

Various components of the system 50 can communicate via or be coupled to via a communication interface, for example, a wired or wireless interface. The communication interface includes, but not limited to, any wired or wireless short-range and long-range communication interfaces. The wired interface can use cables, umbilicals, and the like. The short-range communication interfaces may be, for example, local area network (LAN), interfaces conforming known communications standard, such as Bluetooth® standard, IEEE 702 standards (e.g., IEEE 702.11), a ZigBee® or similar specification, such as those based on the IEEE 702.15.4 standard, or other public or proprietary wireless protocol. The long-range communication interfaces may be, for example, wide area network (WAN), cellular network interfaces, satellite communication interfaces, etc. The communication interface may be either within a private computer network, such as intranet, or on a public computer network, such as the internet.

As will be explained in greater detail elsewhere herein, the various embodiments of the present disclosure, and in particular the distal assembly 150, employ novel structural features to improve the clinical performance as well as enhance the manufacturability of the ablation catheter 105. In particular, the distal assembly 150 includes an insulator preform to, among other things, support and locate the tip electrode and the adjacent ring electrode, as well as operate to electrically insulate the various electrical components of the distal assembly 150.

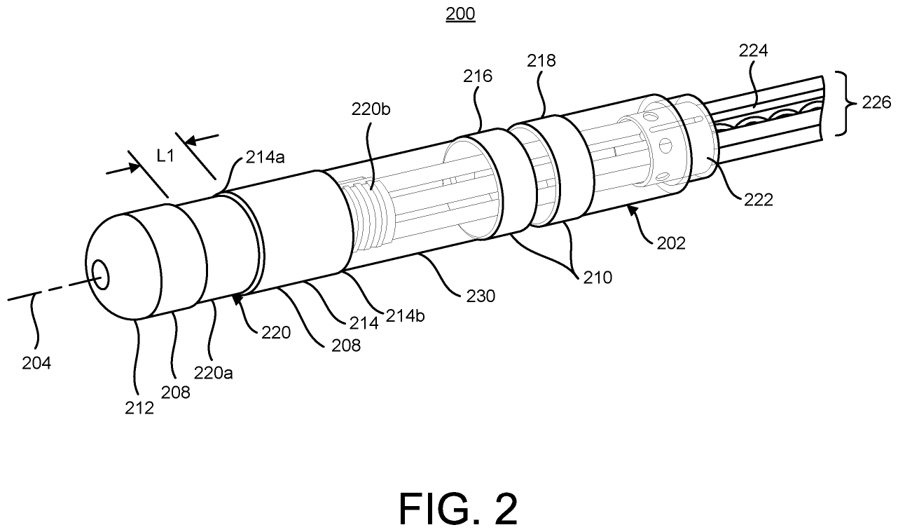

FIG. 2 is an isometric illustration of a distal portion of a cardiac ablation catheter 200. In embodiments, the cardiac ablation catheter 200 corresponds to the ablation catheter 105 depicted in FIG. 1, and includes a distal assembly 202.

As shown, the distal assembly 202 is disposed axially along a longitudinal axis 204 defined by the shaft (not shown in FIG. 2) of the ablation catheter 200. The distal assembly 202 includes a pair of electrodes 208 including a tip electrode 212 and a ring electrode 214, the tip electrode 212 being located at the distal end of the distal assembly 202, and the ring electrode 214 located proximal of and spaced apart from the tip electrode 212. As shown, the ring electrode has a distal leading end 214a and a proximal trailing end 214b. In embodiments, the distal assembly 202 may include additional electrodes, e.g., an additional pair of electrodes 210 including electrodes 216, 218 disposed proximally of and longitudinally spaced from the electrodes 212 and 214. More or fewer electrodes may be employed in other embodiments within the scope of the present disclosure.

The particular operation of the various electrodes (or electrode pairs) can vary depending on the particular clinical use of the ablation catheter 200. In embodiments, the electrodes 212, 214, 216 and 218 may be configured to operate as ablation electrodes, sensing electrodes, or both. For example, any or all of the electrodes 212, 214, 216 and 218 can be configured to be operable for the delivery of ablative energy to target tissue. Additionally, or alternatively, any or all of the electrodes 212, 214, 216 and 218 can be operable as sensing electrodes configured to sense electrical signals (e.g., intrinsic cardiac activation signals and/or electric fields generated by injected currents for use in impedance-based location tracking, tissue proximity or contact sensing, and the like). In one embodiment, the pair of electrodes 208 may be configured to operate as ablation electrodes, e.g., for bi-polar delivery of ablation energy, and in particular, pulsed-field ablation energy for focal ablation of cardiac tissue. In embodiments, the electrodes 216, 218 may be operable as sensing electrodes, or alternatively, as ablation electrodes. In some instances, the second pair of electrodes 210 is configured to measure local impedance, and may act as location sensors for sensing local electric fields in 5 degrees of freedom (e.g., 5 different motions—x, y, z, acceleration, and rotation). In embodiments, except as specifically described herein, the electrodes 212, 214, 216 and 218 may be configured in accordance with those described in co-pending and commonly-assigned U.S. Pat. App. 63/194,716, which is hereby incorporated by reference in its entirety.

In one exemplary embodiment, the electrode pair 212 may be activated with a first polarity, and the electrode pair 210 can be activated with a second polarity opposite the first polarity, so as to define an ablation vector and corresponding electric field therebetween.

It is emphasized, however, that the present disclosure is not limited to the particular electrode configurations and number of electrodes depicted in FIG. 2. Rather, the skilled artisan will appreciate that additional variations of electrode configurations, numbers of electrodes, and the like may be employed within the scope of the present disclosure.

In the illustrated embodiment, the distal assembly 202 further includes a steering ring 222 located at the proximal end of the distal assembly 202. The steering ring 222 is mechanically connected to one or more steering wires 224 connected to a steering mechanism located in the handle (not shown) of the ablation catheter 200, and configured to allow a user to steer the catheter 200 during operation. It is emphasized that the particular steering ring 222 shown in FIG. 2 is for illustration only and in no way limiting. In general, mechanisms for implementing steerability or deflectability to ablation catheters is well known, and thus the skilled artisan will recognize that a wide range of steering technologies can be employed in the ablation catheter 200.

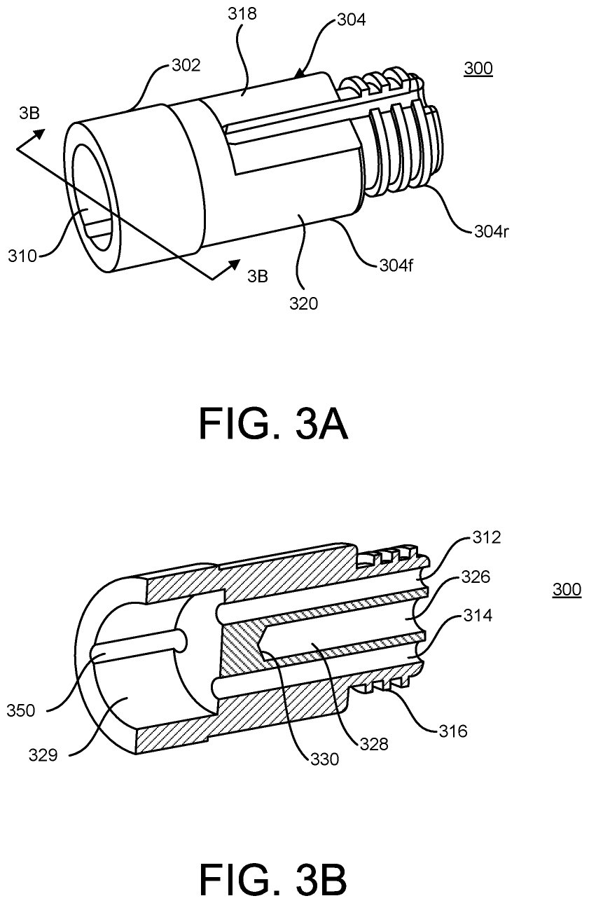

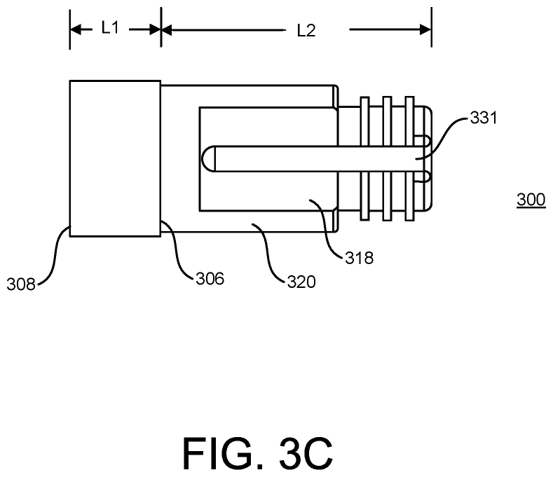

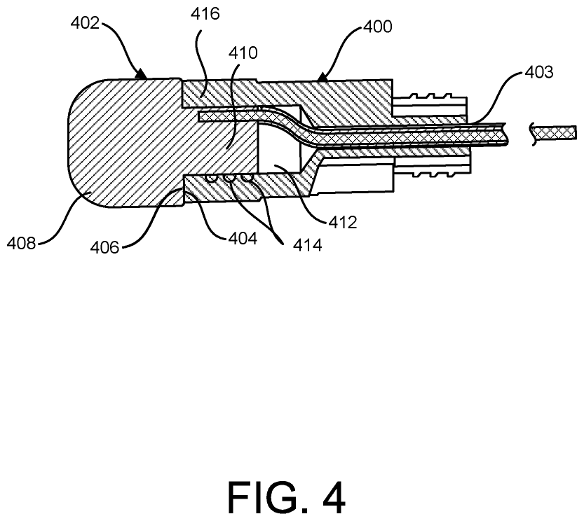

In embodiments, as shown, the distal assembly 202 includes an insulator preform 220, a portion of which is located between the tip electrode 212 and the ring electrode 216. In the illustrated embodiment, the insulator preform 220 includes a distal portion 220a and a proximal portion 220b (partially illustrated in FIG. 2), As shown, the distal portion 220a defines a distal portion length L1. As further shown, the distal portion 220a of the preform 220 is disposed between the tip electrode 212, and the ring electrode 214 is disposed over part of the proximal portion 220b such that the distal leading end 214a of the ring electrode 214 abuts a radial shoulder (shown in FIGS. 3A-3C and FIG. 4) of the insulator preform 220. The distal portion length L1 defines a longitudinal spacing along the longitudinal axis 204 in between the tip electrode 212 and the distal leading end 214a of the ring electrode 214. In embodiments, the insulator preform 220 provides an insulation layer between the conductive surfaces and wires at high potential to each other within the tip region of the catheter. In various embodiments, the length L1 is in the range of about 0.5-4.5 mm. In certain embodiments, the length L1 is from about 1-4 mm. In certain embodiments, the length L1 is from about 2.5 mm to about 3.5 mm. In some embodiments, the length L1 is from about 1-2 mm.

In some instances, the insulator preform provides means for routing conductive wires 226 through the distal assembly 202. In some instances, the insulator preform provides positive placement features for tip components to allow for better component spacing and fitment into subsequent process steps (i.e. mold fit). In some instances, the insulator preform provides protection for components of the cardiac ablation catheter 200, such as navigational sensors or thermocouples, during various use conditions.

In embodiments, the distal assembly 202 further includes an insulating material 230 disposed at least proximally of the ring electrode 214 and encapsulating and forming an outer insulative surface of the distal assembly 202. In embodiments, the insulating material 230 is disposed between the ring electrode 214 and 216. In embodiments, the insulating material 230 is formed by an overmolding process. Alternatively, the insulating material 230 can be formed using a reflow process in which one or more tubular segments of insulating material are disposed about the partially-assembled distal assembly 202 and then heated, as is known in the art. In embodiments, employing an overmolding process to provide the insulating material 230 can have certain advantages, e.g., to reduce or even eliminate the need for subsequent processing (such as the injection of medical adhesive to complete the assembly process and provide a fluid-tight connections between the various components). The insulating material may be commercially available Pebax® 55D and Pelathane® 55D. Both materials may be used in an overmolding process and bonded to an “epoxy bondable” wire insulation. Pellethane may adhere to the tip insulator using primer (e.g. Sivate™ E610) and plasma. Pebax may adhere to the tip insulator using adhesive (e.g. Thermedics 1-MP) without plasma.

FIGS. 3A-3C are isometric, cross-sectional isometric, and plan view illustrations, respectively, of an embodiment of an insulator preform 300 used in the distal assembly of the cardiac ablation catheter of FIG. 1, according to embodiments of the present disclosure.

As shown, the insulator preform 300 includes a distal portion 302 having a distal portion length L1, and a proximal portion 304 having a diameter d2 and a proximal portion length L2. In the illustrated embodiment, the proximal portion 304 has a forward portion 304f and a rearward portion 304r that extends proximally relative to the forward portion 304f. In embodiments, the rearward portion 304r may be omitted.

As shown, the distal portion 302 extends distally from the forward portion 304f of the proximal portion 304, and has a maximum diameter d1. Additionally, the forward portion 304f of the proximal portion 304 has a maximum diameter d2. As further shown, the diameter d1 of the distal portion 302 is greater than the diameter d2 of the forward portion 304f of the proximal portion 304, so as to define a radial shoulder 306 at the intersection of the distal portion 302 and the forward portion 304f of the proximal portion. As further discussed elsewhere herein, the forward portion 304f is dimensioned such that a ring electrode (e.g., the ring electrode 214 in FIG. 2) can be disposed thereover.

In embodiments, the distal portion 302 of the preform 300 is generally cylindrical and includes a distal face 308 and a distal opening 310 in the distal face 308, with an interior of the distal portion 302 defining a distal portion cavity 329. In the illustrated embodiment, the preform 300 includes longitudinal channels 312 and 314 extending through the forward portion 304f of the proximal portion 304 to the distal portion 302. When present as in the embodiment of FIGS. 3A-3C, the longitudinal channels 312 and 314 are designed to facilitate the transmission of overmolding material or adhesive material through forward portion 304f and into the distal portion cavity 329 to enhance the mechanical attachment of a tip electrode to the insulator preform 300 during manufacture of the distal assembly of the ablation catheter. In embodiments, one or both of the longitudinal channels 312 and 314 can be omitted.

In some embodiments as shown, the rearward portion 304r of the proximal portion 304 has a smaller diameter than the forward portion 304f, and includes one or more ribs 316. When present, the ribs 316 operate to enhance mechanical retention to the overmolding resin (i.e., the insulating material discussed in FIG. 2) or medical adhesive during the manufacture of the distal assembly.

In embodiments, the insulator preform 300 includes various structural features to facilitate the positioning and orientation of electrodes, such as the tip electrode 212 and the ring electrode 214 of FIG. 2, as well as the connection of conductor wires to the respective electrodes. In the illustrated embodiment, the forward portion 304f of the proximal portion 304 includes a cut-out flat region 318 and a proximal wire slot 331 to allow for attachment of a conductor wire to the ring electrode 214 (FIG. 2) as well as and overmolding resin (i.e., the insulating material discussed in FIG. 2) overflow. The cut-out flat region 318 is located in a ring electrode landing zone 320 of the forward portion 304f of the proximal portion 304 so as to facilitate alignment of the ring electrode and the tip electrode. In embodiments, the ring electrode landing zone 320 may be dimensioned to allow overmolding resin (i.e., the insulating material discussed in FIG. 2) to flow under the proximal end of the ring electrode to help create a robust connection. Similarly, in the illustrated embodiment, the preform 300 includes a distal wire slot 350 extending along the inside of the distal portion 302 to facilitate connection of a conductor wire to a tip electrode. In embodiments, in addition to accommodating the aforementioned electrical connections, the proximal and distal wire slots 331, 350 may aid in orienting the respective electrodes with respect to the preform 300. In the illustrated embodiment, the proximal portion 304 of the insulator preform 300 may include a proximal opening 326 and a navigator sensor lumen 328 extending from the proximal opening 326 and terminating in closed end 330 so as to form a blind hole. When present, the navigator sensor lumen 328 can be sized and configured to receive a magnetic tracking sensor to enable magnetic localization of the corresponding ablation catheter. In still other embodiments, the preform 300 may include additional structural features to accommodate additional sensors (e.g., temperature sensors, pressure sensors, and the like) and corresponding electrical conductors. It is emphasized, however, that the inclusion of the aforementioned wire and sensor-accommodating and orientation features is not a requirement of the present disclosure, and in embodiments some or all of these features may be omitted or configured differently than in the particular exemplary embodiment shown.

In the various embodiments, and with reference to FIG. 2, the distal portion length L1 is selected to precisely define the desired spacing between a trailing, proximal edge of the tip electrode 212 and the leading, distal edge of the ring electrode 214 while at the same time providing the necessary electrical insulation between the two electrodes. The disclosed configuration thus advantageously provides for consistent, precise electrode spacing, which can be particularly advantageous for use in pulsed electric field ablation catheters.

In the embodiment of FIGS. 3A-3C, the proximal portion length L2 is selected to optimize the overall stiffness of the corresponding distal assembly and stress concentrations at the ring electrode disposed over the proximal portion 304, while still providing adequate structural material to accommodate attachment to the catheter shaft. Additionally, the reduced diameter rearward portion 304r of the proximal portion 304 provides sufficient spacing for overmolding resin (i.e., the insulating material discussed in FIG. 2) to flow around the various conductor wires to reduce fluid leak during the overmolding process.

In the various embodiments, insulator preform 300 may be made of any suitable biocompatible insulative material (e.g., plastic, ceramic, etc.) providing the desired structural and dielectric properties as required for the particular clinical application of the cardiac ablation catheter. In embodiments, the preform 300 can be pre-fabricated using any number of manufacturing process, e.g., may be machined, molded, cast, or manufactured through an additive manufacturing process. In embodiments, exemplary materials used for the preform 300 include, without limitation, polycarbonate, which is transparent to allow for UV cure adhesive, and is also machinable. The insulative material of the insulator preform 300 guarantees dielectric insulation by isolating the tip electrode, ring electrode and navigational sensor (when present) and other electrical components independent of the overmolding resin or reflowed insulation (i.e., the insulating material discussed in FIG. 2).

FIG. 4 is a cross-sectional elevation view of a portion of a distal assembly corresponding to the distal assembly 202 of FIG. 2 in a partially assembled state. As shown, the distal assembly 202 includes an insulator preform 400 and a tip electrode 402 secured thereto, and an electrical conductor wire 403 is secured to the tip electrode 402. As illustrated, the tip electrode 402 extends distally from the distal face 404 of the insulator preform 400. The tip electrode 402 includes a tip electrode shoulder 406 that abuts a distal face 404 of the insulator preform 400. The tip electrode 402 has an active portion 408 having an active portion diameter d3 and a tip electrode shank 410 having a tip electrode shank diameter that is smaller than the active portion diameter. The tip electrode shank 410 is received within a distal opening 412 in the distal face 404 of the insulator preform 400. As described above, the tip electrode shank 410 is secured within the distal portion of the insulator preform 400 via overmolded insulative material and/or adhesive. In embodiments, the tip electrode shank 410 includes one or more radial projections 414 that abut an inner surface of the insulator preform distal portion 416. During the overmolding process, the insulating material encapsulates the radial projections 414 to secure the tip electrode 402 to the insulator preform 400.

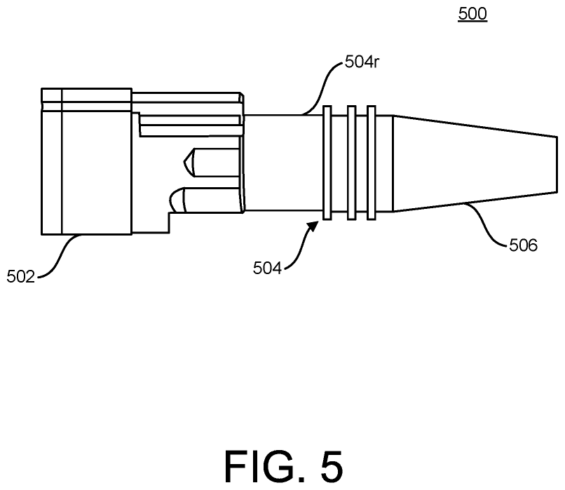

FIG. 5 is an elevation view of an insulator preform 500 for use in the distal assembly of the cardiac ablation catheter of FIG. 1, according to alternative embodiments of the present disclosure. In general, the insulator preform 500 is substantially identical to the insulator preform 300 described above except as described in connection with FIG. 5. Accordingly, the insulator preform 500 includes a distal portion 502 and a proximal portion 504 having a rearward portion 504r that includes a proximal tapered portion 506. The tapered portion 506 may operate to provide strain relief, e.g., to reduce stress between the preform 500 and the overmolding material used to encapsulate the various internal components of the distal assembly in the final product.

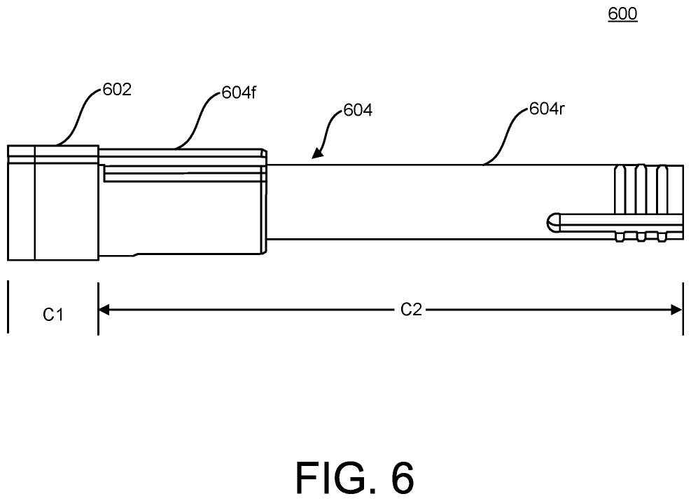

FIG. 6 is an elevation view of an insulator preform 600 for use in the distal assembly of the cardiac ablation catheter of FIG. 1, according to alternative embodiments of the present disclosure. In general, as shown, the insulator preform 600 is substantially identical to the insulator preform 300 described above except as described in connection with FIG. 6. Accordingly, the insulator preform 600 includes a distal portion 602 with a distal portion length L1 and a proximal portion 604 with a proximal portion length L2. As in the insulator preform 300, the proximal portion of the insulator preform 600 includes a forward portion 604f and a rearward portion 304r. The proximal portion length L2 is relatively longer in this embodiment compared to what was shown in FIGS. 3A-3C, by virtue of a relatively elongated rearward portion 604r as shown. The relatively longer proximal portion 304 provides increased capacity for routing of wires and inclusion of orientation features and support of additional components, e.g., sensors and the like, as well as providing increased surface area for attaching the distal assembly to the catheter shaft, e.g., via the aforementioned overmolding process.

The various embodiments of the distal assembly 202 (FIG. 2) can incorporate a range of distal tip electrodes 212. As will be appreciated by the skilled artisan, the distal tip electrode 212 may advantageously be constructed of, or include, materials that are visible under fluoroscopy (i.e., radiopaque materials) to aid the clinician in locating the tip electrode 212, and by extension, other electrodes when present, inside the patient anatomy. One commonly-used material is a platinum-irridium (Pt/Ir) alloy that is both radiopaque and electrically conductive so as to allow the material to deliver ablative energy to target tissue. At the same time, Pt/Ir is a relatively expensive material, and thus it may be advantageous to minimize the volume of such material in the tip electrode 212.

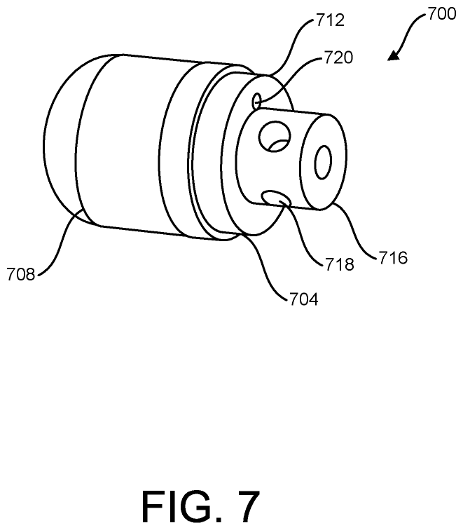

FIG. 7 is an isometric illustration of one exemplary tip electrode 700 that can correspond to the tip electrode 212 of FIG. 2. As shown in FIG. 7, the tip electrode 700 is a two-piece construction having a body 704 and an active shell 708 disposed over a portion of the body 704. As further shown, the body 704 has a proximal portion 712 and an opposite distal portion (not visible in FIG. 7), with the shell 708 disposed over the distal portion. As further shown, extending from the proximal portion 712 is a shank 716 having a plurality of apertures 718. In embodiments, the shank 716 can be received within the distal portion of the various insulator preforms described elsewhere herein to facility assembly of the tip electrode 700 to the insulator preform. Additionally, the apertures 718, when present, can receive attachment materials, e.g., medical adhesive or overmolding material to enhance the mechanical attachment of the tip electrode 700 to the insulator preform. As will be appreciated by the skilled artisan and as describe elsewhere herein, the apertures 718 are merely one illustrative example of such securement-enhancing features that may be employed, but are not required in all embodiments of the disclosure.

In one embodiment, the body 704 is made of a relatively inexpensive, non-metallic material, e.g., a ceramic, while the shell 708 can be formed partly or entirely of a radiopaque and electrically conductive material to facilitate visualization of the tip electrode 700 under fluoroscopy and also operate as an active electrode portion for delivery of ablative energy. The aforementioned construction provides the required electrode functionality while at the same time minimizing the volume of relatively expensive radiopaque material (e.g., Pt/Ir) needed for the radiopaque active portion. Because the body 704 is not electrically conductive, the embodiment of FIG. 7 includes one or more vias 720 extending through the body 704 to facilitate electrical connection of the active shell 708 and conductive wires (not shown) for delivery of ablative energy to the shell 708. In other embodiments, the body 704 may be formed of an electrically-conductive material (e.g., titanium) that has minimal or no radiopacity but is relatively less expensive than the radiopaque materials such as Pt/Ir. In such embodiments, the vias 720 can be omitted and the required electrical connection(s) to the tip electrode 700 can be made directly to the body 704 (e.g., by attaching the conductive wire(s) directly to the shank 716).

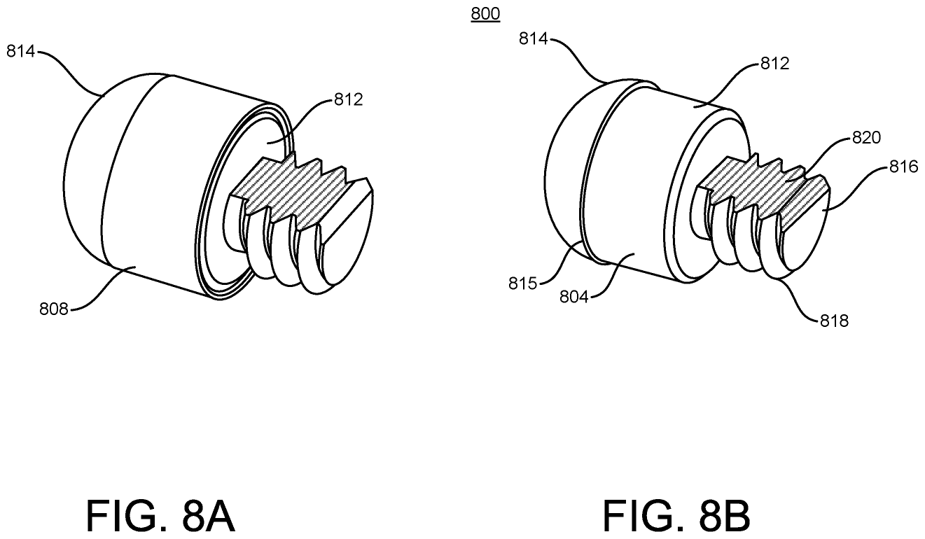

FIGS. 8A and 8B are isometric illustrations of an alternative tip electrode 800 that can correspond to the tip electrode 212 of FIG. 2 according to some embodiments. Similar to the tip electrode 700, the tip electrode 800 is a two-piece construction that includes a body 804 and a radiopaque ring 808. FIG. 8A depicts the assembled tip electrode 800 while FIG. 8B depicts only the body 804 without the radiopaque ring 808.

As shown, the body 804 has a proximal portion 812 and an opposite distal portion 814. As can be seen in FIG. 8A, the proximal portion 812 has a diameter that is smaller than the maximum diameter of the distal portion 814 so as to define a shoulder 815 at a proximal end of the distal portion 814. In the assembled tip electrode 800, the radiopaque ring 808 is disposed over the proximal portion 812 and abuts the shoulder 815. As a result, the assembled tip electrode 800 is substantially isodiametric, i.e., the maximum diameters of the distal portion 814 and the radiopaque ring 808 are substantially the same so as to minimize discontinuities between the distal portion 814 and the radiopaque ring 808.

As further shown, the body 804 includes a shank 816 extending from the proximal portion 812 having a plurality of ribs 818. In embodiments, the shank 816 can be received within the distal portion of the various insulator preforms described elsewhere herein to facility assembly of the tip electrode 800 to the insulator preform. Additionally, the ribs 818, when present, can provide increased surface area for attaching the shank 816 to the insulator preform, e.g., using medical adhesive or overmolding material, so as to enhance the mechanical attachment of the tip electrode 800 to the insulator preform. As further shown, in the illustrated embodiment, the shank 816 includes a flat region 820 to facilitate attachment of a conductor wire to the tip electrode 800.

In embodiments, the body 804 is of a single-piece solid construction formed of an electrically-conductive but relatively inexpensive material, e.g., titanium, and the radiopaque ring 808 can be formed from an electrically-conductive and radiopaque material such as Pt/Ir. Similar to the tip electrode 700 described above, the design of the tip electrode 800 provides the desired visibility under fluoroscopy while reducing the volume of relatively expensive radiopaque material utilized. In embodiments, the radiopaque ring 808 can be attached to the body 804 using conventional manufacturing techniques, e.g., welding.

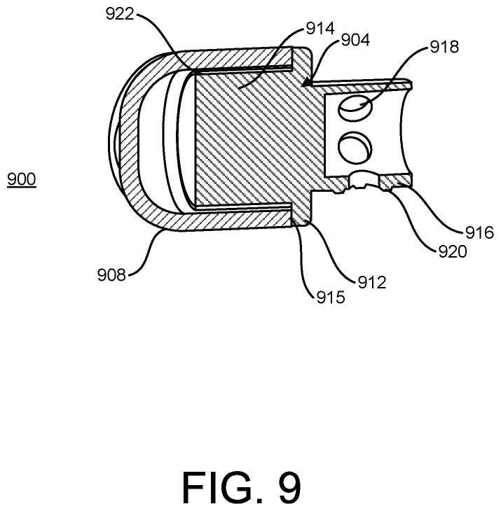

FIG. 9 is a cross-sectional perspective illustration of an alternative tip electrode 900 that can correspond to the tip electrode 212 of FIG. 2 according to some embodiments. As shown, the tip electrode 900 includes a body 904, and an outer shell 908 that together form the electrically-active portion of the tip electrode 900. As further shown, the body 904 includes a proximal portion 912 and a distal portion 914, and the shell 908 is disposed over the distal portion 914. The proximal portion 912 has a maximum diameter greater than the maximum diameter of the distal portion 914 so as to define a shoulder 915 against which the proximal end of the distal portion 914 abuts in the assembled tip electrode 900. As a result, the assembled tip electrode 900 is substantially isodiametric at the junction of the proximal and distal portions 912, 914, i.e., the maximum diameters of the distal portion 914 and the proximal portion 912 are substantially the same so as to minimize discontinuities between the distal portion 914 and the proximal portion 812.

As further shown, the body 904 includes a shank 916 extending from the proximal portion 912 having a plurality of apertures 918 as well as ribs 920. In embodiments, the shank 916 can be received within the distal portion of the various insulator preforms described elsewhere herein to facility assembly of the tip electrode 800 to the insulator preform. Additionally, the apertures 918 and ribs 920, when present, can provide increased surface area for attaching the shank 916 to the insulator preform, e.g., using medical adhesive or overmolding material, so as to enhance the mechanical attachment of the tip electrode 900 to the insulator preform. In embodiments, the shank 916 may also include features (not shown in FIG. 9) to locate and attach a conductor wire to the tip electrode 900.

In the embodiment of FIG. 9, the tip electrode 900 further includes a radiopaque ring 922 disposed between the distal portion 914 of the body 904 and an inner surface of the shell 908. In embodiments, the body 904 may be constructed from an electrically-conductive but relatively inexpensive material, e.g., titanium, and the radiopaque ring 922 can be formed from an electrically-conductive and radiopaque material such as Pt/Ir. Similar to the tip electrodes 700 and 800 described above, the design of the tip electrode 900 provides the desired visibility under fluoroscopy while reducing the volume of relatively expensive radiopaque material utilized. In embodiments, the shell 908 and the radiopaque ring 922 can be attached to the body 904 using conventional manufacturing techniques, e.g., welding.

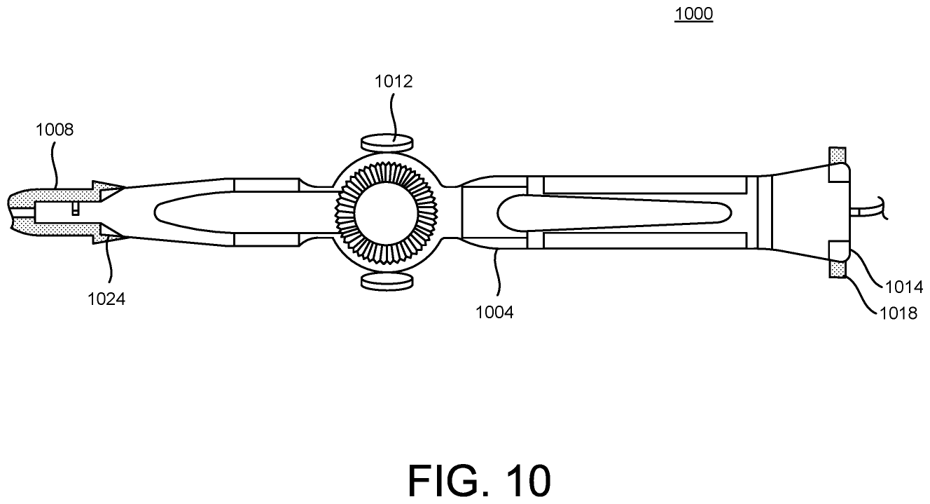

FIG. 10 is an elevation plan view of a handle 1000 that can correspond to the handle 105a of the cardiac ablation catheter 105 of FIG. 1, according to embodiments of the present disclosure. As described elsewhere herein, the cardiac ablation catheter 105 (FIG. 1) can be a deflectable or steerable catheter whereby the distal portion of the shaft can be selectively deflected or steered by the clinician as needed for the particular medical procedure being performed. The handle 1000 can, in many respects, be of conventional design suitable for use in deflectable ablation catheters, with the exception of enhancements to accommodate the relatively high direct-current voltages (e.g., in excess of 1000 V) used for pulsed field ablation applications. As shown in FIG. 10, the handle 1000 includes a housing 1004 that is configured to be gripped by the clinician, and a nose portion 1008 that operates as a transition section through which the catheter shaft and working components, e.g., electrical conductors, steering wires and the like extend. In the illustrated embodiment, the handle 1000 further includes a bi-wing deflection knob 1012 that is rotatable by the clinician to deflect the distal portion of the shaft in a manner known in the art. Additionally, the proximal end 1014 of the handle 1000 is adapted, via a connector assembly 1018 (shown schematically in FIG. 10), to allow the cardiac ablation catheter 105 to be operatively coupled to the electroporation generator 130 (FIG. 1) and other components of the overall electrophysiology system 50 (FIG. 1).

In embodiments, the housing 1004 may be constructed of two or more shell pieces that are joined together to enclose the various functional components disposed therewithin. The interfaces between the handle shell portions, the nose portion 1008, the deflection knob 1012 and the connector assembly 1018 may, in conventional catheter handle designs, include gaps that could create leakage pathways into the interior of the handle 1000. In various embodiments of the disclosure, the aforementioned interfaces are sealed with an epoxy potting material, e.g., as illustratively shown at 1024, to fill, or substantially fill, these gaps in the handle housing. In this way, potential points of leakage into the interior of the handle that could be bridged by saline, moisture and the like can be substantially sealed. In some embodiments, high voltage electrode wires (i.e., wires electrically coupled to ablation electrodes for delivery of high-voltage PFA pulses) are routed through the handle 1000 to individual pinouts (not shown) for further electrical isolation from electrically erasable programmable read-only memory (EEPROM), navigation sensor wires, and other low voltage circuits.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. A cardiac ablation catheter comprising:
a handle;
an elongated shaft having a proximal end and a distal end, the proximal end extending distally from the handle; and
a distal assembly having a proximal end and a distal end, the proximal end secured to the distal end of the shaft, the distal assembly comprising:
a tip electrode at the distal end of the distal assembly;
a first ring electrode located proximal of and spaced apart from the tip electrode, the first ring electrode having a distal leading end and a proximal trailing end; and
an insulator preform comprising a proximal portion having a forward portion defining a first diameter, and a distal portion extending distally from the proximal portion, the distal portion having a distal face and a second diameter greater than the first diameter so as to define a radial shoulder, and a distal portion length,
wherein the tip electrode extends distally from the distal face of the insulator preform, and the first ring electrode is disposed over the proximal portion such that the distal leading end of the first ring electrode abuts the radial shoulder of the insulator preform, and wherein the distal portion length defines a longitudinal spacing between the tip electrode and the first ring electrode distal leading end.

2. The cardiac ablation catheter of claim 1, wherein the tip electrode includes a tip electrode shoulder that abuts the distal face of the insulator preform.

3. The cardiac ablation catheter of claim 2, wherein the distal portion of the insulator preform includes a distal opening in the distal face.

4. The cardiac ablation catheter of claim 3, wherein the tip electrode includes an active portion having an active portion diameter, and a tip electrode shank having a tip electrode shank diameter that is smaller than the active portion diameter, and wherein the tip electrode shank is received within the distal opening in the distal face of the insulator preform.

5. The cardiac ablation catheter of claim 4, wherein the distal assembly further comprises a second ring electrode located proximally of and longitudinally spaced from the first ring electrode.

6. The cardiac ablation catheter of claim 5, wherein the distal assembly further comprises an insulating material disposed at least proximally of the first ring electrode.

7. The cardiac ablation catheter of claim 6, wherein the insulating material is disposed between the first ring electrode and the second ring electrode.

8. The cardiac ablation catheter of claim 6, wherein the insulator preform includes first and second longitudinal channels extending through the proximal portion to the insulator preform distal portion.

9. The cardiac ablation catheter of claim 8, wherein the insulating material extends through the first and second longitudinal channels and about the tip electrode shank so as to secure the tip electrode to the insulator preform.

10. The cardiac ablation catheter of claim 8, wherein the tip electrode shank includes a plurality of radial projections that abut an inner surface of the insulator preform distal portion, and wherein the insulating material encapsulates the radial projections to secure the tip electrode to the insulator preform.

11. The cardiac ablation catheter of claim 7, wherein the tip electrode shank includes a plurality of radial apertures extending inward, and wherein the insulating material extends through the radial apertures to secure the insulator preform to the distal assembly.

12. The cardiac ablation catheter of claim 11, wherein the insulator preform proximal portion includes a proximal opening and a navigator sensor lumen extending from the proximal opening and terminating in blind hole.

13. The cardiac ablation catheter of claim 12, wherein the insulator preform proximal portion has a planar portion defining a space to accommodate attachment of an electrical conductor to the first ring electrode.

14. An ablation electrode assembly for a pulsed field ablation catheter, the ablation electrode assembly comprising:
an insulator preform comprising a proximal portion having a forward portion defining a first diameter, and a distal portion extending distally from the proximal portion, the distal portion having a distal face and a second diameter greater than the first diameter so as to define a radial shoulder, and a distal portion length;
a tip electrode extending distally from the distal face of the insulator preform; and
a ring electrode disposed over the proximal portion of the insulator preform such that a distal leading end of the ring electrode abuts the radial shoulder of the insulator preform, and wherein the distal portion length of the insulator preform defines a longitudinal spacing between the tip electrode and the first ring electrode distal leading end.

15. The ablation electrode assembly of claim 14, wherein the tip electrode includes a tip electrode shoulder that abuts the distal face of the insulator preform.

16. The ablation electrode assembly of 15, wherein the distal portion of the insulator preform includes a distal opening in the distal face.

17. The ablation electrode assembly of claim 16, wherein the tip electrode includes an active portion having an active portion diameter, and a tip electrode shank having a tip electrode shank diameter that is smaller than the active portion diameter, and wherein the tip electrode shank is received within the distal opening in the distal face of the insulator preform.

18. The cardiac ablation catheter of claim 14, wherein the insulator preform includes first and second longitudinal channels extending through the proximal portion to the insulator preform distal portion, and wherein an insulating material extends through the first and second longitudinal channels and about the tip electrode shank so as to secure the tip electrode to the insulator preform.

19. A method of making an ablation electrode assembly of a cardiac ablation catheter, the method comprising:
providing an insulator preform comprising a proximal portion having a forward portion defining a first diameter, and a distal portion extending distally from the proximal portion, the distal portion having a distal face having a distal opening, a second diameter greater than the first diameter so as to define a radial shoulder, and a distal portion length;
securing a tip electrode to the distal portion of the insulator preform so that the tip electrode extends distally from the distal face of the insulator preform; and
securing a ring electrode over the proximal portion of the insulator preform such that a distal leading end of the ring electrode abuts the radial shoulder of the insulator preform, wherein the distal portion length of the insulator preform defines a longitudinal spacing between the tip electrode and the first ring electrode distal leading end.

20. The method of claim 19, wherein the tip electrode includes an active portion having an active portion diameter, and a tip electrode shank having a tip electrode shank diameter that is smaller than the active portion diameter, and wherein securing the tip electrode to the insulator preform includes inserting the tip electrode shank within the distal opening in the distal face of the insulator preform.
